(19) United States
(12) Patent Application Publication
(10) Pub. No.: US 2024/0101337 A1
(43) Pub. Date: Mar. 28, 2024

(54) DELIVERY HEAD FOR A FLUID DISPENSER AND FLUID DISPENSER WITH SUCH A DELIVERY HEAD

(71) Applicant: Aptar Radolfzell GmbH, Radolfzell (DE)

(72) Inventor: Jürgen GREINER-PERTH, Gottmadingen (DE)

(21) Appl. No.: 18/466,920

(22) Filed: Sep. 14, 2023

(30) Foreign Application Priority Data
Sep. 16, 2022 (EP) 22196192.3

(51) Int. Cl. B65D 83/40; A61M 15/00; A61M 15/08; B05B 11/10; B05B 15/40; B65D 83/28

(52) U.S. Cl. CPC B65D 83/40; A61M 15/009; A61M 15/08; B05B 11/1042; B05B 15/40; B65D 83/28

(57) ABSTRACT

A delivery head for a fluid dispenser for coupling to a fluid store. The delivery head has a nozzle unit with a plurality of fine nozzle openings with a maximum clear cross-section of 0.02 mm². The protective cap is configured to encourage the fluid to remain in the nozzle openings for a long time, or to remove the fluid from the nozzle openings in a targeted fashion.

### CROSS-REFERENCE TO RELATED APPLICATION

This claims priority from European Application No. 22196192.3, filed Sep. 16, 2022, the disclosure of which is hereby incorporated by reference in its entirety.

### TECHNICAL FIELD

The invention concerns a delivery head for a fluid dispenser, and a fluid dispenser with a fluid store and a delivery head of the type according to the invention.

### BACKGROUND AND SUMMARY

The delivery head of a generic fluid dispenser and of a fluid dispenser according to the invention is configured for coupling or attachment to a fluid store. The delivery head includes a nozzle unit with a plurality of fine nozzle openings, each with a maximum clear, free or open cross-section of 0.02 mm², through which fluid can be discharged from the fluid store to a surrounding atmosphere.

Such a delivery head is configured for breaking down the fluid into fine droplets on discharge through the very fine nozzle openings, so as to form a spray mist. In particular, such a form of delivery may be used for fluids to be applied to the user's eye, nose or mouth. The use of a plurality of nozzle openings leads to a particularly fine mist. In particular, this achieves a mean droplet size which would not be achievable by an eddy chamber in the region of the delivery outlet of the delivery head. The small droplet size is advantageous for example for being able to transport fluid in the form of a fluid mist into the region of the user's bronchi or lungs.

It has however been found that the use of nozzle openings may entail the problem that after use, residual fluid dries in the nozzle openings and residue remains which can later obstruct the further delivery of fluid through the nozzle openings, so that on later use, the creation of the desired fine droplets may be hindered. Depending on type of residue, the process may even be irreversible. The delivery of further fluid then does not lead to dissolution of the residue, but the creation of droplets is permanently hindered. As well as such deposits, depending on the fluid and the material surrounding the nozzle openings, corrosion may also occur which also counters the proper creation of mist in subsequent delivery processes.

The invention provides a delivery head and a fluid dispenser with such a delivery head in which the risk of such deterioration in delivery is reduced.

According to the invention, a delivery head and a fluid dispenser are proposed. In generic fashion, these have a plurality of fine nozzle openings with a clear, free or open cross-section of maximum 0.02 mm², through which fluid can be discharged from a fluid store into a surrounding atmosphere. The nozzles may be oriented in parallel. Preferably however, it is provided that the nozzle openings diverge, in order thereby to form a widening spray cone.

The nozzle unit preferably has a nozzle plate through which nozzle openings are made. For example, it may be a metallic nozzle plate which was produced by galvanic cutting, but other materials and manufacturing processes are also possible. The nozzle plate may be flat. To generate a widening spray cone however, a curved design may be provided, in particular with slightly diverging orientation of the nozzle openings.

The nozzle unit preferably has at least 10 nozzle openings, in particular preferably at least 25 nozzle openings. The nozzle openings are preferably arranged in a two-dimensional structure, for example in a matrix form or in concentric circles. Preferably, as well as a nozzle plate, the nozzle unit comprises a separately produced carrier, in particular in the manner of a sleeve. The carrier may in particular be designed as a plastic carrier. The nozzle plate is inserted in the carrier. The carrier facilitates handling during mounting and fixing to a supporting housing part of the delivery head.

Preferably, at least one filter is connected upstream of the nozzle unit for eliminating particles from the fluid so that they cannot block the nozzle openings. If a carrier is used to support the nozzle plate, this preferably carries the at least one filter. The filter may be designed as a membrane filter or as a deep filter. Preferably, a cascade of several filters is provided, each of which has different filtration limits.

The nozzle unit forms the end of the delivery path along which the fluid passes from the fluid store of the corresponding dispenser into the environment. Preferably, an outlet valve, which at the same time may be the outlet valve of a pump device of the delivery head, is connected upstream of the nozzle unit. This outlet valve opens under positive pressure and closes when this positive pressure is eliminated. Therefore with such a design, after the end of actuation, fluid which has already entered the region of the nozzle unit cannot be drawn back.

As cited initially, a delivery head of the described type has the problem that fluid remaining in the region of the nozzle openings may, when not in use, lead to deposits or corrosion in or on the nozzle openings, which influence the spray picture and hence reduce the effect of the fluid delivery on later use.

In order to counter this, it is proposed that the delivery head has a removable and refittable protective cap which, when fitted, isolates the nozzle unit from a surrounding atmosphere, and which is designed to reduce the risk of deposits and corrosion.

According to a possible embodiment, it is provided that the protective cap defines an isolation space which adjoins the nozzle unit on the outside and only has a small inner volume. This is a maximum of 5 ml, preferably however less, in particular a maximum of 3 ml.

The effect of this isolation space is to counter the evaporation of fluid. The majority of the fluid thus remains inside the nozzle openings, and the deposits formed on drying are thus prevented or reduced. The smaller the isolation space formed by the cap, the less evaporation occurs.

As already explained, an outlet valve is preferably connected upstream of the nozzle unit. Preferably, the volume in the fluid path between the outlet valve and nozzle openings is a maximum of 5 ml, in particular a maximum of 2 ml, so that here again almost no space is left which could prevent evaporation of the fluid from the nozzle openings or into which fluid could in some cases slowly run back.

Alternatively or additionally, it is also provided that the protective cap has a contact face which is spaced from an output side end of the nozzle openings by a maximum of 1 mm, in particular preferably however lies directly against the nozzle unit when the protective cap is fitted.

This contact face prevents or at least reduces the fluid's tendency to evaporate in the cap interior. This also encourages the fluid to remain in the nozzle openings so that no harmful deposits occur here.

To form the isolation space with small interior volume, in particular it may be provided that an inner sealing surface of the protective cap lies circumferentially against an outer face of the applicator on which the nozzle unit is provided. In particular, for this the protective cap may have a circumferential web on the inside, which is raised relative to the remainder of the cap inner face and preferably has the form of a circular cylindrical or slightly conical ring wall. During fitting or screwing of the protective cap, this lies against a preferably also slightly conical outer face of the applicator and seals this. It may be advantageous here if a separate sealing element is provided on the inner wall, for example in the form of an elastic sealing ring or other body made of TPE material. This may ensure a particularly good seal.

Said inner wall, where applicable without said sealing element, may be an integral part of a unitary cap body which also forms the other walls of the cap and the coupling faces for coupling to a housing of the dispenser. It is however also conceivable that the protective cap has an inner cap element which forms the isolation space, and which accordingly may rest in particular against the outer faces of the applicator in order to create the isolation space. This inner cap element is not integrally connected to the outer cap element.

The inner cap element and the outer cap element are preferably fixedly connected together. However, designs are also conceivable in which the two cap elements are movably connected to one another. In particular, the inner cap element may be arranged rotatably on the outer cap element. This for example allows provision of a screw cap in which the outer cap element is provided with a thread, wherein then the inner cap element, which creates the seal of the isolation space when the cap is screwed on, may after contact with the applicator remain rotationally stationary relative thereto. A design is also possible in which the inner cap element and the outer cap element are completely separate and can be handled separately.

As explained initially, the isolation space can greatly limit the volume which could allow evaporation of the fluid from the nozzle openings, so that the fluid remains in the nozzle openings. This is extremely helpful in particular when the fluid would lead to harmful deposits in the case of evaporation, but its remaining in the nozzle openings would not lead to corrosion.

Insofar as the combination of the fluid of the fluid dispenser and the material of the nozzle unit, in particular the nozzle plate, carries a risk of corrosion, it may however be advantageous to remove the fluid from the nozzle openings.

For this, a design of a protective cap is proposed in which the protective cap is adapted to the shape of the applicator such that the isolation space delimited by the protective cap and the applicator, when fitted in an intermediate position, is closed by the circumferential contact of the protective cap on the applicator, and then undergoes a reduction. This is achieved for example in that in an end position when the protective cap is fully fitted, the isolation space has an inner volume which amounts to less than 70% of the size of the inner volume of the isolation space in the intermediate position, preferably less than 60%, or even less than 50%.

Such a reduction of the isolation space compresses the air present therein when the protective cap is applied. The pressure of the fluid in the nozzle openings then rises so that, on transition from the intermediate position to the end position, air inside the isolation space penetrates into the nozzle openings and displaces the fluid from there. If an outlet valve is provided upstream of the nozzle openings, it is an advantage if this can be opened by the external positive pressure achievable by means of the protective cap, and the fluid can thereby be pushed back into the fluid store.

A further possibility for rapid removal of the fluid from the nozzle openings lies in a design in which a moisture-absorbing element is arranged inside the isolation space. Here, this may be a sponge-like or otherwise porous body, in particular with hydrophilic properties. Particularly preferred are elements made of cellulose.

The moisture-absorbing element may serve the purpose of reducing the moisture in the air and hence allowing rapid evaporation. For this, the moisture-absorbing element need not necessarily be in very close contact with the nozzle unit. However, a design is advantageous in which the moisture-absorbing element lies directly against the nozzle openings and absorbs the fluid evenly from the nozzle openings. This avoids deposits.

The use of a moisture-absorbing element is therefore advantageous in particular when the fluid adversely affects the delivery characteristic of the nozzle opening not primarily because of deposits but because of corrosion.

An aspect of the invention also concerns, as well as a design with a cap with a low-volume isolation space and/or a contact face close to the nozzle openings, a design with a potentially larger cap interior, wherein however in the above-described fashion, a moisture-absorbing element is provided.

The described moisture-absorbing element need not be provided in the immediate vicinity of the nozzle unit. It is however advantageous if the element is arranged close to the nozzle unit or even lies thereon. It has been found that a distance of less than 0.5 mm, in particular a distance of 0.2 mm or even a distance of less than 0.1 mm, allows a particularly rapid removal of fluid from the nozzle openings. In the case of a small distance, the fluid does not evaporate but is removed from the nozzle openings and in particular from an outside of the nozzle plate. Accordingly, the risk of deposits is thereby reduced.

It is possible to connect the moisture-absorbing element to a surrounding atmosphere via a connecting channel which passes through the cap wall. In this way, the moisture can be discharged to the environment. Such a connecting channel preferably penetrates through an end face of the protective cap, and is in particular preferably covered by the moisture-absorbing element.

A delivery head may in particular be configured such that it has a base against which an actuating unit can be pressed down along a main axis for the purpose of actuation, wherein the nozzle openings have a mean orientation in the direction of the main axis. In particular, the nozzle openings are preferably provided at a distal end of an applicator such as a nasal applicator which is part of the actuating unit.

In such a case, the protective cap is preferably provided for fitting or screwing in the direction of the main axis, so that in this way the above-mentioned isolation space is closed or the fluid-absorbing element is brought closer to the nozzle openings and preferably lies against these when the cap is fully fitted.

Another design of a delivery head provides that the delivery head has a base against which an actuating unit can be pressed down along a main axis for the purpose of delivery, wherein in this case the nozzle openings are angled relative to the main axis. Preferably, a mean orientation of the nozzle openings encloses with the main axis an angle between 70° and 110°. With such a design, relative to the main axis and main extent direction of the fluid dispenser, an approximately sideways delivery of the fluid is thus provided.

In such a case, a protective cap may be used which can be fitted on the housing of the delivery head in the direction of the main axis. Since it is thereby difficult to position a fluid-absorbing element, or the wall of an isolation space, at the correct position relative to the nozzle openings in the fitted situation, with angled configurations of main axis and mean extent direction of the nozzle openings, it is preferred if the protective cap is provided for fitting in a joining direction which is angled relative to the main axis, preferably in the direction of the mean extent direction of the nozzle opening. In such a case, the cap is also placed sideways onto the delivery head.

A delivery head is preferably provided for coupling to a fluid store, in particular in that the base of the delivery head is fitted onto a fluid store by means of a snap connection or threaded connection. An integral connection of the base and fluid store is also possible.

The delivery head in particular preferably has an actuating unit which is displaceable relative to the base, in particular can be pressed down in the direction of the base. This actuation movement leads to the fluid being conveyed from the fluid store to the nozzle unit and hence to fluid delivery. There is a possibility that the fluid is conveyed by means of a pump device with a volumetrically variable pump chamber which has a valve on the input side and on the output side. Another possibility is that the fluid is stored under pressure in the fluid store and the delivery head has an outlet valve which can be opened by the actuating unit, so that the pressurised fluid is pushed to the nozzle unit and discharged there.

Preferably, the actuating unit is actuated via a manual actuation face which may be provided on a distal end of the actuating unit. A design with a lateral and possibly circumferential finger support is also possible.

A fluid dispenser equipped with a delivery head of the described type comprises a fluid store, preferably with an inner volume of less than 200 ml, in particular with an inner volume of less than 100 ml. In filled state, it is preferably filled with a cosmetic or pharmaceutical fluid.

### BRIEF DESCRIPTION OF THE DRAWINGS

Further advantages and aspects of the invention arise from the claims and from the following description of preferred exemplary embodiments of the invention, which are explained below with reference to the figures.

FIGS. 1A, 1B and 1C show a first exemplary embodiment of a dispenser according to the invention.

FIGS. 2A, 2B and 2C show, for a further exemplary embodiment, the sequence of fitting a protective cap.

FIGS. 3A and 3B show a further exemplary embodiment, and FIG. 3C shows a variant thereof.

FIGS. 4A, 4B and 4C show a further exemplary embodiment.

FIGS. 5A, 5B and 5C show a further exemplary embodiment and a particular variant thereof is shown in FIG. 5C.

FIGS. 6A, 6B, 7A and 7B show two further dispensers according to the invention.

FIGS. 8A, 8B and 8C show a final exemplary embodiment of the invention.

### DETAILED DESCRIPTION

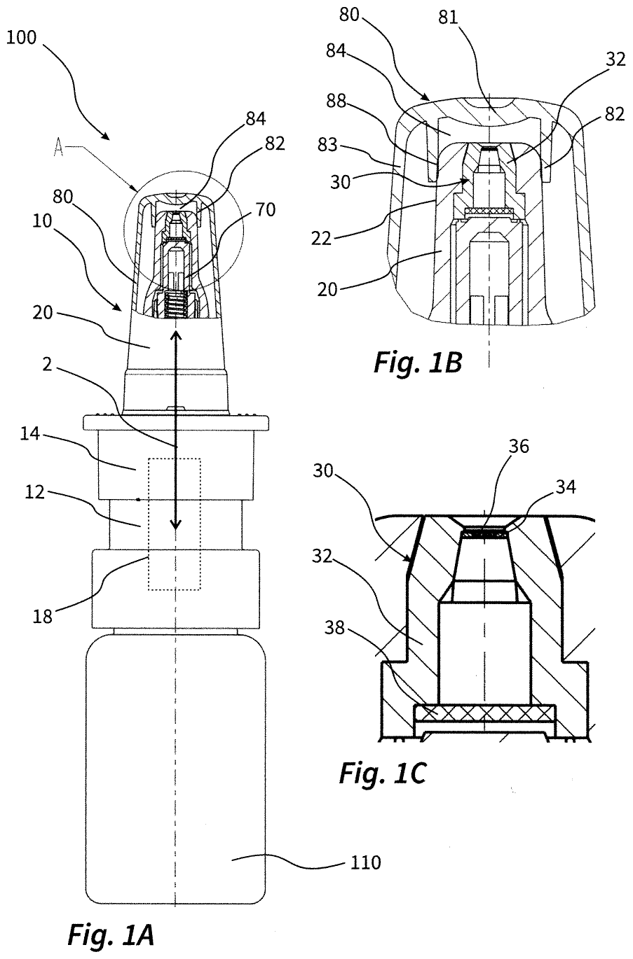

FIGS. 1A to 1C show a first variant of a fluid dispenser 100 according to the invention with a fluid store 110 and a delivery head 10 attached thereto.

The delivery head 10 has a base 12 on which the fluid store 110 is attached, and an actuating unit 14 which is displaceable against a spring force relative to this base 12 in the direction of the arrow 2, and has a finger contact face 16 and an applicator 20, in this case an elongate, slender nasal applicator 20.

By pressing down the actuating unit 14, a pump device 18 (not shown in detail) is actuated which conveys fluid from the fluid store 110 to a nozzle unit 30 at the distal end of the nasal applicator 20. When the fluid store 110 is configured as a pressure store, as an alternative to the pump device, a valve device 18 may be provided which is opened when the actuating unit 14 is pressed down and then allows the stored pressurised fluid to flow to the nozzle unit 30.

In the design of FIGS. 1A to 1C, an outlet valve with a spring-loaded valve body 70 is arranged upstream of the nozzle unit 30. This outlet valve opens when an internal fluid pressure reaches an opening pressure, and the valve body 70 is accordingly displaced downward against the force of the compression spring.

The fluid dispenser 100 has a protective cap 80 which is fitted to the actuating unit 14 and in the present exemplary embodiment is configured as a push-on cap.

The nozzle unit 30 of the fluid dispenser 100 is shown in enlarged illustration in FIG. 1C. The nozzle unit 30 has a sleeve-like plastic carrier 32 which is attached at the upper end of the applicator 20 inside an end opening. The plastic carrier 32 surrounds a supply channel, the internal end of which contains a filter 38 and the external end of which is provided with a nozzle plate 34. The nozzle plate has a plurality of fine nozzle openings 36. The nozzle units 30 of the further exemplary embodiments described below have a corresponding structure.

As evident from FIG. 1B, the protective cap has an end face 81 and an outer casing face 83. Also, a circumferential web 82 is provided on the inside of the end face 81 and, when the protective cap 80 is fitted, comes into circumferential contact with a distal end of the nasal applicator 20 and thereby, together with the end face of the applicator 20 and the nozzle unit 30 arranged there, defines an isolation space 84. The inner volume of this isolation space is around 2 ml.

When the fluid dispenser has been used, some of the fluid remains inside the nozzle openings 36 after use. Depending on the nature of the fluid and the material of the nozzle plate 34, this may not be critical. If the fluid delivered by means of the fluid dispenser 100 is however configured such that, on evaporation, deposits remain e.g. crystallised salts, this can negatively influence the spray picture in subsequent applications.

The very small isolation space 84 defined by the protective cap 80 effectively prevents the fluid from evaporating in the nozzle openings 36 after use when the protective cap 80 is fitted. Thus when not in use, the fluid remains in the nozzle openings 36 for at least a few hours or days without deposits forming.

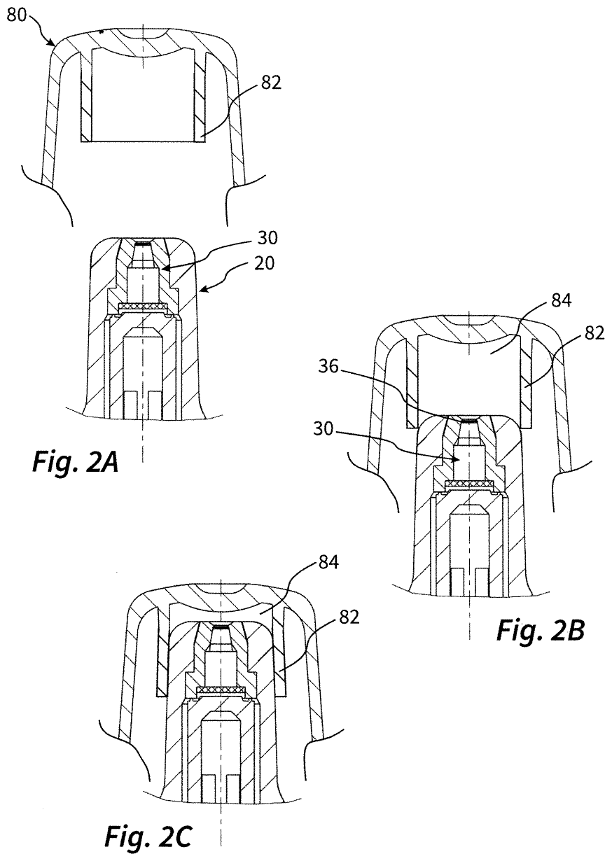

An externally similar structure is illustrated in FIGS. 2A to 2C. Despite the similarity with the design in FIGS. 1A to 1C, the function here is however different.

In the design of FIGS. 2A to 2C, the protective cap 80 is provided with a web 82 which is particularly long in the fitting direction. This means that the interior 84 delimited by the circumferential web 82 is already isolated from the remaining cap interior in the intermediate state of FIG. 2B during fitting of the protective cap 80. As the protective cap 80 is pushed further on, up to the final state in FIG. 2C, the inner volume of the interior 84 reduces significantly, in the present case by around 80% to 90%. Accordingly, the air present therein is compressed and the air pressure increases, so that this air is expelled from the outside into the nozzle openings 36 of the nozzle unit 30 where it displaces inward the fluid previously present therein. This reduces the risk of corrosion occurring during a storage phase following use.

The spring of the valve body 70d of the dispenser 100 according to FIGS. 2A to 2C is preferably designed such that the pressure rise caused in the interior 84 is sufficient to open the valve so that fluid can flow back into the dispenser.

The design of the fluid dispenser in FIGS. 2A and 2B differs from the design in FIGS. 1A to 2C with respect to the protective cap 80. In this design, there is no isolation space. Instead, on the inside of the protective cap 80, a fluid-absorbing element 86 is provided, in particular as a cellulose body. This cellulose body is carried by a carrier ring 82 and is arranged such that when the cap is fitted, it is arranged at a slight distance from the nozzle plate 34 and the nozzle openings 36. On a side pointing away from the nozzle plate 34, the carrier ring 82 is interrupted by a connecting channel 90 such that the fluid absorbed by the fluid-absorbing element 86 can be discharged outward into the surrounding cap interior.

In this design, the purpose of the cap design is not to guarantee that fluid remains in the nozzle openings. Rather, the cap promotes rapid drying since evaporated fluid is absorbed in the fluid-absorbing element 86, which prevents a rise in air humidity between the fluid-absorbing element 86 and nozzle plate 34. To further accelerate the removal of fluid, the fluid-absorbing element 86 may lie gap-free against the nozzle plate 34.

The use of such a design, which promotes the rapid removal of fluid from the nozzle openings, like the design in FIGS. 2A to 2C, is particularly suitable when the fluid develops a corrosive effect on the nozzle plate 34. This may be the case for example with metal nozzle plates. In such cases, the main problem is not deposits of fluid constituents on the nozzle openings 36, but corrosion caused by the fluid remaining in the nozzle openings 36.

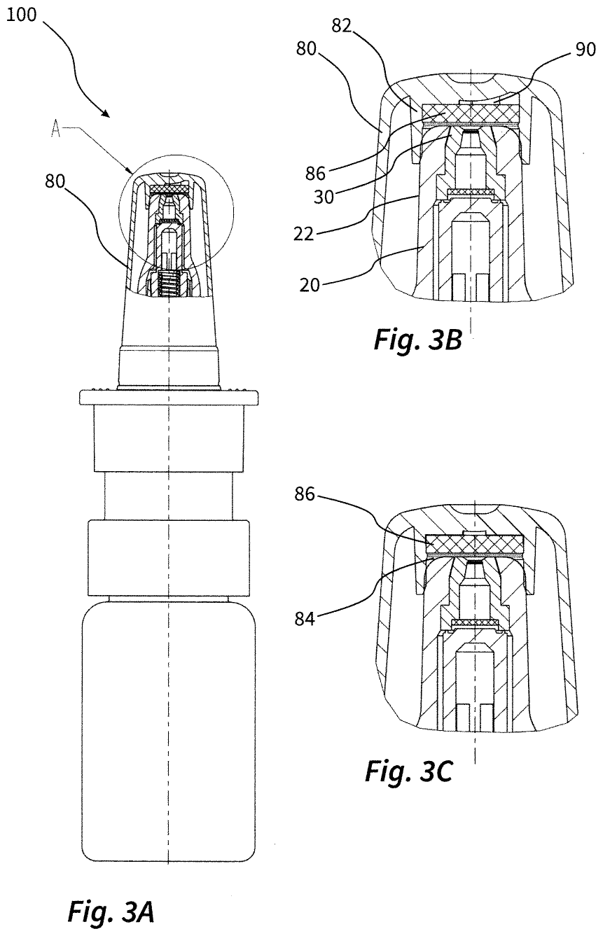

In the design according to FIGS. 3A and 3B, said connecting channel 90 is provided, by means of which fluid can be discharged from the fluid-absorbing element 86 into the rest of the cap interior. FIG. 3C shows an alternative in which this connecting channel 90 is not provided. The design therefore has a closed isolation space 84, corresponding to the design in FIGS. 1A to 1C, which in comparison therewith is merely supplemented by the fluid-absorbing element 86.

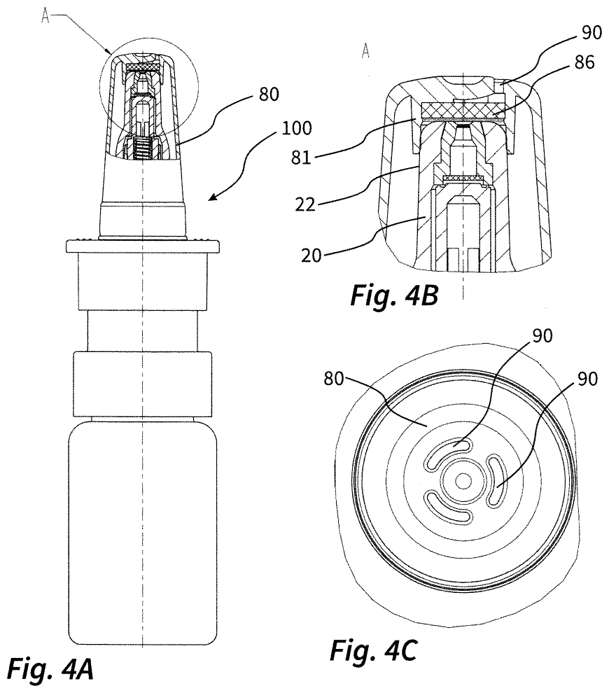

In the design of FIGS. 4A to 4C, it is again provided that rapid drying of the fluid is promoted by a fluid-absorbing element 86. In contrast to the preceding design however, here it is provided that the connecting channel 90 connects the fluid-absorbing element 86 not to another part of the cap interior but to an environment. For this, in the manner shown in FIG. 4C, in total three openings are provided in the end face of the cap which form the connecting channels 90.

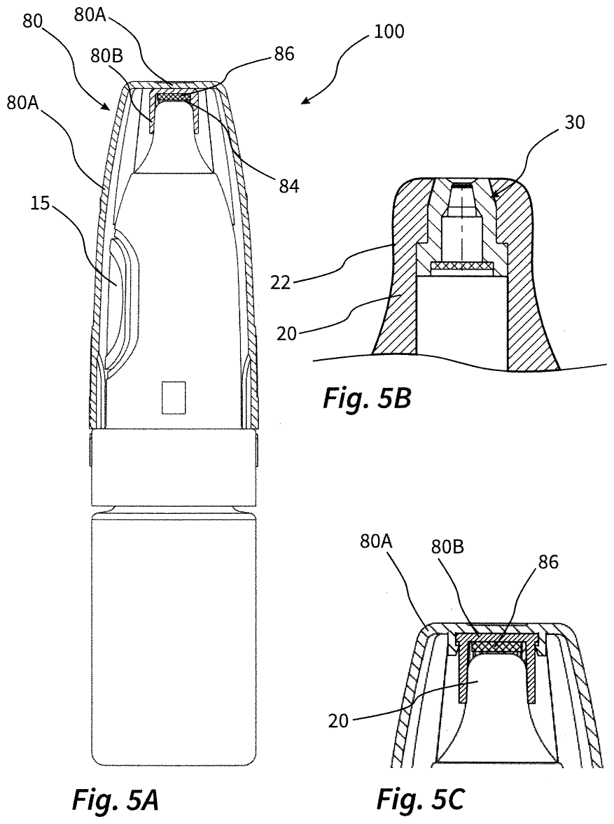

FIGS. 5A to 5C show a further exemplary embodiment and a derived variant thereof. In this design, the delivery device itself is structured slightly differently since it has a side actuation delivery device with a side actuation button 15. Here there is no pressing down of an actuating unit on which the applicator is provided.

Corresponding to the preceding exemplary embodiment, the fluid dispenser 100 of FIG. 4 has a nasal applicator 20 in which a nozzle unit 30 corresponding to that in FIGS. 1A to 1C is provided, as shown in the sectional illustration of FIG. 5B. As also provided in the design of FIGS. 1A to 1C, when the protective cap 80 is fitted, an isolation space 84 in the region of the nozzle unit 30 defines a small volume. The specific feature here however is that this isolation space 84 is formed by an inner cap element 80B together with the applicator, wherein this inner cap element 80B is formed separately from an outer cap element 80A which constitutes the main element of the protective cap 80 and is mechanically coupled to the housing of the discharge device when the protective cap 80 is fitted.

The inner cap element 80B may be fixedly connected to the outer cap element 80A so that they can be handled jointly and function as a mutually permanently stationary element.

The inner cap element 80B may alternatively also be configured as a completely separate cap element 80A. Removal of the protective cap 80 by removing its main constituent 80A in such a case does not yet cause removal of the inner cap element 80B. This must later be moved separately.

A third possibility is shown in FIG. 5C. Here, the inner cap element 80B is connected to the outer cap element 80A via a latch connection, so that these are always handled jointly. However, the inner cap element 80B remains rotatable relative to the outer cap element 80A. This may be particularly advantageous if the protective cap 80 or its outer cap element is attached to the discharge device via a thread, and accordingly the protective cap 80 is turned during fitting. Here, the remaining rotational mobility of the cap element 80B relative to the outer cap element 80A allows the rotational movement of the inner cap element 80B to be terminated after contact of the cap element 80B with the applicator 20 and, on continued screwing on of the outer cap element 80A, the cap elements 80A, 80B accordingly turn relative to one another.

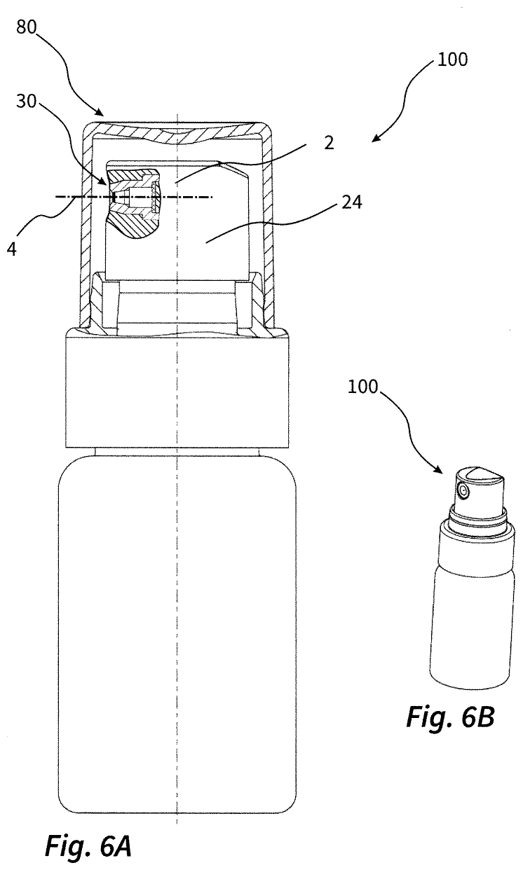

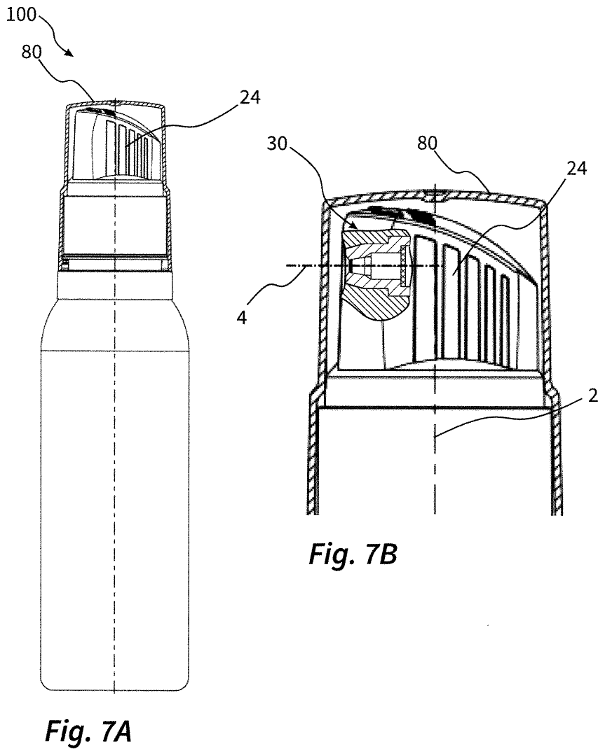

FIGS. 6A, 6B and 7A, 7B show further fluid dispensers 100 with nozzle units 30 through which fluid can be discharged to an environment, wherein by deviation from the preceding designs, an actuating direction 2 encloses an angle of around 90° with a mean delivery direction 4 which is defined by the extent direction of the nozzle openings 36. The nozzle unit 30 is provided on a press-down actuating device 24 by means of which a pump device of the dispenser is actuated.

In this design too, a protective cap 80 is provided which, when fitted, closes tightly against an environment and thus reduces the risk that all the fluid will evaporate from the nozzle openings 36 of the nozzle unit 30 and leave disadvantageous residue.

The fundamental structure of the design of FIGS. 7A and 7B resembles that of FIGS. 6A and 6B. Here however, particular value is placed on the fact that the protective cap 80 surrounds the actuating device 24 with nozzle unit 30 as closely as possible, so that the volume of the cap interior when fitted is small. This guarantees that only a very small quantity of the fluid remaining in the nozzle openings 36 can evaporate until the resulting air humidity in the cap interior prevents further evaporation.

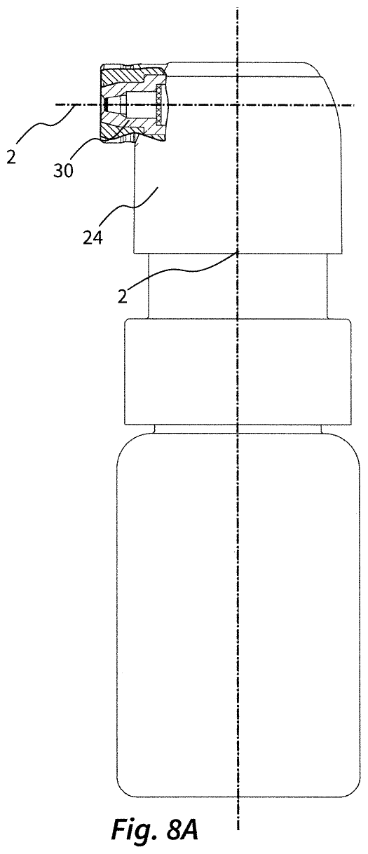

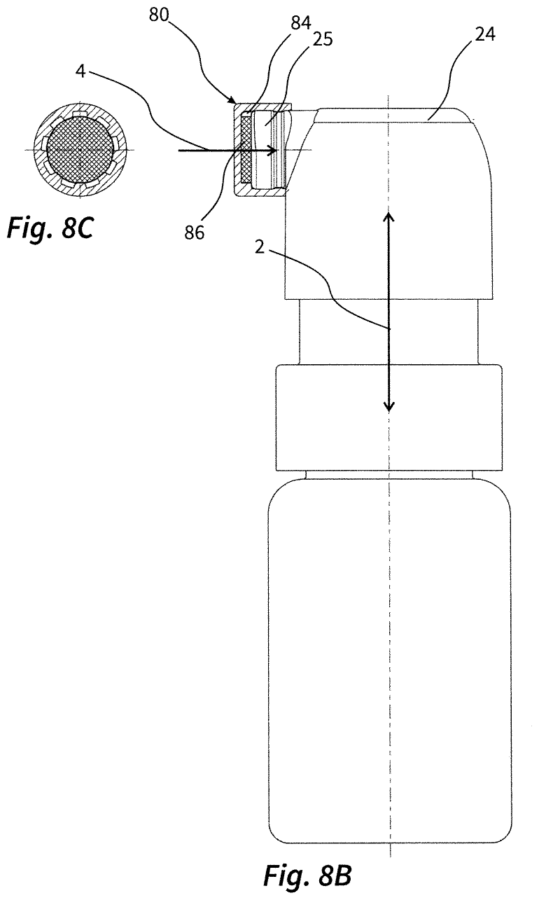

FIGS. 8A to 8C show a further version of a fluid dispenser 100 according to the invention with a delivery head according to the invention. Like the designs of FIG. 6A to 7B, here again an actuating device 24 is provided which is pressed down for the purpose of fluid discharge. The nozzle unit 30 is provided on this actuating device and has a mean discharge direction 2 which stands at an angle of around 90° to the actuation direction.

As FIG. 7B shows, the protective cap 80 is here designed differently from FIG. 6A to 7B, in that it is fitted in the direction of the mean discharge direction 2, i.e. from the side. The actuating device has a laterally protruding extension 25 inside which the nozzle unit 30 is arranged. The outside of this extension 25 is provided with a circumferential depression so that the protective cap 80 can be snap-fitted therein.

In fitted state, the protective cap 80 with the applicator extension 25 defines an isolation space 84 with small inner volume. A moisture-absorbing element 86 is arranged inside this isolation space 84 and largely fills the isolation space and, in particular in the fitted state of FIG. 7B, directly adjoins this nozzle unit 30. The moisture-absorbing element 86 is therefore able, after fitting, to absorb fluid directly from the nozzle unit 30 and in particular extract this from the nozzle openings 36 of the nozzle unit 30.

1. A delivery head for a fluid dispenser comprising:
a nozzle unit with a plurality of fine nozzle openings, each nozzle opening having a maximum free cross-section of 0.02 mm², through which free cross-section fluid is discharged from the fluid store to a surrounding atmosphere; and
a removable and refittable protective cap which protective cap, when fitted, isolates the nozzle unit from a surrounding atmosphere,
the protective cap defining an isolation space, the isolation space, when the protective cap is fitted, adjoining the nozzle unit and having a maximum inner volume of 5 ml, and the delivery head is configured for coupling to a fluid store.

2. The delivery head according to claim 1, further including an applicator having a distal end and an outer face,
the nozzle unit being provided at the distal end of the applicator, and
pthe protective cap has an inner sealing surface, the inner sealing surface lying against the outer face of the applicator.

3. The delivery head according to claim 1, wherein
the protective cap has an inner cap element forming the isolation space, and
an outer cap element, the inner cap element being arranged inside the outer cap element.

4. The delivery head according to claim 2, wherein
the protective cap has on an inside a circumferential web, the circumferential web, when fitted, resting circumferentially on the applicator of the delivery head and delimiting the isolation space on the outside.

5. The delivery head according to claim 2, wherein
the protective cap is adapted to a shape of the applicator of the delivery head such that the isolation space delimited by the protective cap and the applicator, when fitted in an intermediate position, is closed by circumferential contact of the protective cap on the applicator, and
in an end position, when the protective cap is fully fitted, the isolation space has an inner volume amounting to less than 70% of a size of the inner volume of the isolation space in the intermediate position, so that a transition from the intermediate position to the end position, air inside the isolation space is compressed and pushed through the nozzle openings into the delivery head.

6. The delivery head according to claim 1, further including
a moisture-absorbing element arranged inside the isolation space.

7. A delivery head for a fluid dispenser, the delivery head comprising:
a nozzle unit with a plurality of fine nozzle openings, each nozzle opening having a maximum free cross-section of 0.02 mm², through which free cross-section fluid is discharged from a fluid store to a surrounding atmosphere; and
a removable and refittable protective cap which protective cap, when fitted, isolates the nozzle unit from a surrounding atmosphere,
the protective cap having a moisture-absorbing element, and the delivery head being configured for coupling to the fluid store.

8. The delivery head according to claim 6, wherein
the moisture-absorbing element and the nozzle unit are separated by an intermediate space with a maximum inner volume of 0.1 ml.

9. The delivery head according to claim 6, wherein
the moisture-absorbing element lies directly against an outside of the nozzle unit in a region of the nozzle openings.

10. The delivery head according to claim 7, wherein the moisture-absorbing element has a side facing away from the nozzle unit, the delivery head further including at least one connecting channel
on the side of the moisture-absorbing element, the at least one connecting channel creating a connection to a cap interior or to a surrounding atmosphere.

11. The delivery head according to claim 6, wherein
the moisture-absorbing element comprises one or both of a porous material and/or cellulose.

12. The delivery head according to claim 1, further comprising
a base and an actuating unit, the actuating unit being pressable downwardly against the base along a main axis, and
the nozzle openings are oriented in a direction of the main axis.

13. The delivery head according to claim 11, further comprising
a base and an actuating unit, the actuating unit being pressable downwardly against the base along a main axis,
the nozzle openings being angled relative to the main axis.

14. The delivery head according to claim 1, wherein:
the nozzle unit has a nozzle plate, the nozzle openings extending through the nozzle plate; and/or
the nozzle unit has at least 25 nozzle openings; and/or
the nozzle unit has a plastic carrier, the nozzle plate being inserted in the plastic carrier; and/or
the nozzle unit has at least one filter disposed upstream of the nozzle openings; and/or
the delivery head further includes an outlet valve arranged upstream of the nozzle unit.

15. A fluid dispenser for delivering a fluid in atomised form, comprising:
a fluid store;
a delivery head configured for coupling to the fluid store, the delivery head comprising:
a nozzle unit with a plurality of fine nozzle openings, each nozzle opening having a maximum free cross-section of 0.02 mm², through which free cross-section fluid is discharged from the fluid store to a surrounding atmosphere; and
a removable and refittable protective cap which protective cap, when fitted, isolates the nozzle unit from a surrounding atmosphere, the protective cap defining an isolation space, the isolation space, when the protective cap is fitted, adjoining the nozzle unit and having a maximum inner volume of 5 ml.

16. A delivery head for a fluid dispenser comprising:
a nozzle unit with a plurality of fine nozzle openings having an output-side end, each nozzle opening having a maximum free cross-section of 0.02 mm², through which free cross-section fluid is discharged from the fluid store to a surrounding atmosphere; and
a removable and refittable protective cap which protective cap, when fitted, isolates the nozzle unit from a surrounding atmosphere, the protective cap having a contact face which contact face, when the protective cap is fitted, lies against the nozzle unit or is spaced from the output-side end of the nozzle openings by a maximum of 1 mm, and the delivery head is configured for coupling to a fluid store.

17. The fluid dispenser according to claim 2, wherein the protective cap has a separate sealing element, the separate sealing element forming the inner sealing surface.

18. The fluid dispenser according to claim 2, wherein the applicator is configured as a nasal applicator.

19. The fluid dispenser according to claim 3, wherein the inner cap element and the outer cap element are fixedly connected together.

20. The delivery head according to claim 13, wherein the nozzle openings are angled relative to the main axis at an angle between 70° and 110°, and the protective cap is attached to the delivery head in a joining direction, the joining direction being angled relative to the main axis.

21. The fluid dispenser according to claim 15, wherein:
the fluid store has a volume of 200 ml or less; and/or
the fluid store is filled with a pharmaceutical or a cosmetic fluid; and/or
the delivery head is connected to the fluid store by a snap or screw connection or the delivery head has a base integrally connected to the fluid store.
